(19) United States
(12) Patent Application Publication
(10) Pub. No.: US 2019/0083005 A1
(43) Pub. Date: Mar. 21, 2019

(54) SYSTEM AND METHOD FOR IMPLEMENTING A CHAIR RISE TEST

(71) Applicant: KONINKLIJKE PHILIPS N.V., EINDHOVEN (NL)

(72) Inventors: Ronaldus Maria Aarts, Geldrop (NL); Christian Andreas Tiemann, Eindhoven (NL)

(73) Assignee: KONINKLIJKE PHILIPS N.V., EINDHOVEN (NL)

(21) Appl. No.: 16/081,445

(22) PCT Filed: Feb. 9, 2017
(86) PCT No.: PCT/EP2017/052936

§ 371 Date: Aug. 31, 2018

(30) Foreign Application Priority Data
Mar. 7, 2016 (EP) 16158943.7

(51) Int. Cl. A61B 5/11; G08B 21/04

(52) U.S. Cl. CPC A61B 5/1116; A61B 2562/0219; G08B 21/0469; G08B 21/0476

(57) ABSTRACT

A monitoring system for monitoring the movement of a subject during toilet use comprises a set of sensors for detecting the presence of the subject in a toilet room, and their sitting or standing posture. A timer determines the duration of a stand-to-sit activity and the duration of a sit-to-stand activity during the toilet use, and this enables information about a chair rise test to be estimated.

### FIELD OF THE INVENTION

This invention relates to a system and method for implementing a chair rise test.

### BACKGROUND OF THE INVENTION

A chair rise test is a physical performance test which is commonly used in geriatric studies. A five times sit-to-stand test (SXSST) is one common example. The test assesses functional lower extremity strength, transitional movements, balance, and fall risk. The test is generally supervised by a physiotherapist and administered in the following way:

(i) The patient sits with their arms folded across the chest and with their back against the chair;

(ii) Therapist gives the instruction to stand up and sit down five times as quickly as the patient is able, after a start command;

(iii) The therapist tracks the duration of the test using a stopwatch. Timing begins when the start instruction is given and ends when the patient touches the chair after the fifth repetition.

The validity and reliability of the test has been assessed in many studies. For example, the article by J. M. Guralnik, L. Ferrucci, C. F. Pieper, S. G. Leveille, K. S. Markides, G. V. Ostir, S. Studenski, L. F. Berkman, and R. B. Wallace, “Lower Extremity Function and Subsequent Disability Consistency Across Studies, Predictive Models, and Value of Gait Speed Alone Compared With the Short Physical Performance Battery”, J. Gerontol. A. Biol. Sci. Med. Sci., vol. 55, no. 4, pp. M221-M231, Jan. 2000 showed that the inability to rise from a chair five times in less than 13.6 seconds is associated with increased disability and morbidity. Another study has found the optimal cut-off time for performing the sit-to-stand test in predicting recurrent fallers to be 15 seconds. Another study has found a cut-off score of 12 seconds to enable discrimination between healthy elderly subjects, and subjects with chronic stroke.

The five times sit-to-stand test is thus a valuable physical performance test. However, it is not pleasant to perform the test on a regular basis as it requires time and effort from the elderly patient. Furthermore, although the test is simple in nature, it requires a physiotherapist to supervise the test and track the duration.

### SUMMARY OF THE INVENTION

The invention is defined by the claims.

According to examples in accordance with an aspect of the invention, there is provided a monitoring system for monitoring the movement of a subject during toilet use, comprising:

a set of sensors for detecting the presence of the subject in a toilet room, and their sitting or standing posture;

a controller adapted to determine the duration of a stand-to-sit activity and the duration of a sit-to-stand activity, wherein the controller is further adapted to estimate a sit-to-stand test performance for the subject based on the duration information; and

a memory for storing the duration information or an output for outputting the duration information to an external device.

This system provides a more natural environment for performing a chair rise test in a daily fashion, and in particular in the home environment. The system does not require additional effort from the elderly subject, nor does it require a physiotherapist. This provides therefore also the opportunity for more frequent testing.

The system emulates a sit-to-stand test in the home environment of the elderly subject by making use of a sit-to-stand activity and stand-to-sit activity which occur naturally during the day. Sitting and standing activities occur frequently and are well-defined in the toilet environment. Furthermore, the test conditions are very similar each time. For instance, the room décor is typically fixed, as well as the toilet position and height, which might not be the case for chairs in the living room for example.

The controller is further adapted to estimate a sit-to-stand test performance for the subject based on the duration information. This may be achieved by extrapolating from individual toilet visits, for example by training the interpretation of the duration information using real sit-to-stand test performance information obtained as a part of a test or calibration procedure. In this way, it is possible to estimate the performance of a user for a sit-to-stand test, such as a three times, five times or ten times sit-to-stand test, from the duration information collected from individual stand-to-sit and sit-to-stand transitions.

The monitoring system may thus simply monitor the individual sit and stand activities at each toilet visit, in order to extrapolate to a sit-to-stand test. However, the system may also allow the subject to perform a test. The subject may either simply decide to perform a sit-to-stand activity every once in a while (e.g. weekly) whenever they remember. The system can then automatically detect this activity and use the duration information as calibration data. Alternatively, the system may have a mechanism for instructing the subject to perform a sit-to-stand test, for example by communicating with a device worn by the subject, such as a smart phone or smart watch.

In some cases, the test performance estimated by the controller is a five times sit-to-stand test performance.

The sensors are preferably mounted in the toilet room, so that they do not need to be worn by the subject. In this way they do not interfere at all with the normal daily living of the subject.

The set of sensors may comprise at least one PIR sensor. PIR sensors can be used for detecting presence, general movement and also direction of movement. For example, the at least one PIR sensor may be for detecting presence in the toilet room and vertical movement.

The set of sensors may further comprise a microphone. This can be used for enhancing the event detection to improve the accuracy of the duration information.

The set of sensors may further comprise a heart rate sensor. This may be used to determine the effort level of the subject, which may again be used to enhance the event detection and thus improve the accuracy of the duration information. It is preferably worn by the subject, but it may instead be a remote sensor.

The set of sensors may further comprise an accelerometer worn or carried by the subject. This may be used to improve the accuracy of detection of the timing at which sitting and standing activities take place. Furthermore, such an accelerometer may also be used to provide an indication of the nature of a standing or sitting activity (smooth or jerky for example) as well as an indication of the amount of effort exerted.

The controller may be further adapted to time the duration of a sitting activity and the duration of a standing activity before and after the sitting activity. While these time durations may not relate to the stand-to-sit or sit-to-stand motions, they may provide additional information about the subject which may be of interest.

The invention also provides a monitoring installation, comprising a monitoring system as defined above installed in a toilet room.

Examples in accordance with a second aspect of the invention comprise a method for monitoring the movement of a subject during toilet use, comprising:

detecting the presence of the subject in a toilet room, and detecting their sitting or standing posture;

determining the duration of a stand-to-sit activity and the duration of a sit-to-stand activity; and

storing the duration information or outputting the duration information to an external device.

This duration information enables a sit-to-stand performance to be estimated without needing the subject to take any special measures to perform the test or to remember to perform the test.

The method may comprise detecting presence in the toilet room and detecting vertical movement. The method may comprise detecting sounds in the toilet room and/or detecting a heart rate of the subject and/or receiving movement information from an accelerometer device carried or worn by the subject. The duration of a sitting activity and the duration of a standing activity before and after the sitting activity may also be timed.

From the duration information, a five times sit-to-stand test performance for the subject is obtained.

The invention may be implemented at least in part in software and the invention also provides a computer program for this purpose.

### BRIEF DESCRIPTION OF THE DRAWINGS

Examples of the invention will now be described in detail with reference to the accompanying drawings, in which:

FIG. 1 shows a toilet room having an installed monitoring system;

FIG. 2 shows the monitoring system;

FIG. 3 shows a monitoring method; and

FIG. 4 shows a generic computer architecture for implementing the processing required.

### DETAILED DESCRIPTION OF THE EMBODIMENTS

The invention provides a monitoring system for monitoring the movement of a subject during toilet use, comprising a set of sensors for detecting the presence of the subject in a toilet room, and their sitting or standing posture. A timer determines the duration of a stand-to-sit activity and the duration of a sit-to-stand activity during the toilet use, and this enables information about a chair rise test to be estimated.

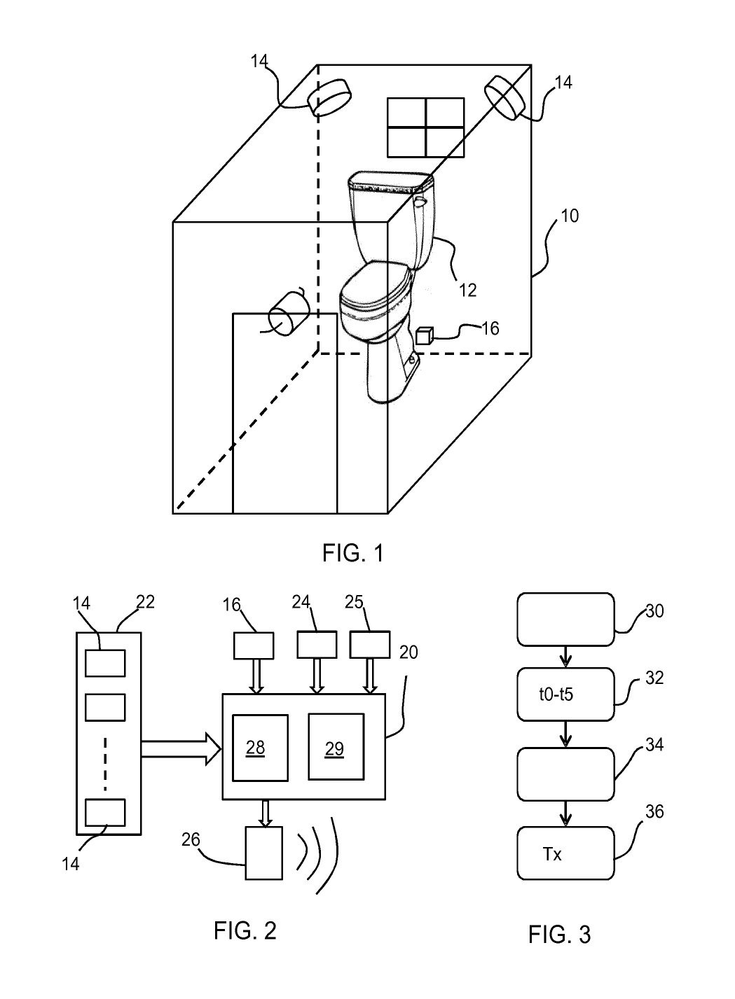

FIG. 1 shows a monitoring system installed into a toilet room 10 (i.e. a closet, or bathroom or shower room). The room includes a toilet 12, and this what is meant by “toilet room”.

A sensor system comprises a set of sensors for detecting sit-to-stand and stand-to-sit activities of a person in the room 10. One option for the sensors is passive infrared (PIR) sensors 14. A microphone 16 may also be used to identify different types of activities such as movements, evacuation, and flushing.

These sensors are mounted in the toilet room, so they are part of the room infrastructure. Thus, they do not need to be worn or carried by the subject. Thus, in most basic and least intrusive implementation, the subject does not need any awareness of the sensors, as they can all be remote sensors provided within the toilet room.

There may be additional sensors worn by the subject. These may be sensors specifically for the monitoring system. However, they may be sensors which the subject is already using for other monitoring purposes.

For example, an unobtrusive heart rate sensor may be used to provide additional indications relating to the acts of standing and sitting based on increases and decreases in heart rate. Sitting and standing may be detected by a steady decrease or increase in heart rate, respectively. In addition, the increase and recovery of the heart rate may provide important information about the fitness level of the elderly subject. The heart rate dynamics can be easily measured with a wrist watch like device and can be correlated with the timing duration values. Non-wearable solutions may also be used measure heart rate such as Doppler radar, or a so-called vital signs camera.

A microphone may be added to improve the accuracy of the system and to obtain more detailed information about the various events during the toilet visit. For instance, a microphone could be used to detect evacuation, hand washing, and flushing. Hence, a microphone could be used to increase the set of events that can be detected and their timings.

An accelerometer may also be worn (or carried) by the subject, for monitoring movement of the subject. The accelerometer may then communicate with the main system controller, for example in the toilet room, or it may communicate with a remote database over the internet (e.g. a cloud). The accelerometer may be provided as a pendant to be worn by the subject. There exist accelerometer pendant devices for wearing by the elderly, such as the Philips Lifeline (trade mark) pendant which forms part of a medical alert system for providing fall detection.

In addition to providing further information for improving the accuracy of detecting the timing of sitting and standing events, an on-body accelerometer of this type also enables an estimation of the power exertion during the sit-to-stand function. This may be made based on the vertical acceleration profile, which is indicative of physical performance.

Furthermore, based on the acceleration signal, it may also be possible to determine whether the subject is moving smoothly in one flow from sit to stand, or whether the movement is less regular (e.g., the subject is using his hands to grasp a kind of railing or another object to aid the standing-up process).

Another sensor which may be used is a pressure sensor on the toilet seat, so that the times at which contact with the toilet seat is made and then broken can be accurately determined.

Based on detection of the different events, the system determines the duration of sit-to-stand and stand-to-sit activities, as well as the total time spent in the toilet room. This duration information can then be transmitted to a device of a caregiver, or stored in memory for later retrieval. The data can then be tracked over time. This enables early detection of any deterioration in the performance of the subject.

FIG. 2 shows the components of the system.

A controller 20 receives the sensor signals from the set 22 of PIR sensors 14, as well as from an optional other sensors, such as a heart rate sensor 24, accelerometer 25 and microphone 16. There may be other additional sensors, as mentioned above. The controller transmits data in this example using a wireless transmitter 26. The controller 20 has a processor 28 and a memory 29. The controller 20 tracks time so that the time at which different events take place may be recorded. For example, this may yield the following timing points, each corresponding to such an “event”:

t0: the time the elderly subject enters the toilet room,

t1: the time the sitting process is started,

t2: the time the elderly subject is fully seated,

t3: the time the elderly subject starts rising (after the use of the toilet is complete),

t4: the time the elderly subject is standing,

t5: the time the elderly subject has left the toilet room.

The timer used to provide a time stamp on these events is based on the clock system of the controller 20.

The events can be detected using a set of PIR sensors. Entering and leaving the room can be detected straightforwardly using standard PIR presence detection. In addition to detection of presence, a PIR sensor may also be used to detect movement, based on changes in infrared radiation due to a changing distance between the person and the sensor. When placed for instance on the ceiling, it becomes be possible to detect vertical movement e.g., standing and sitting.

A typical PIR sensor comprises two detectors of which the signals are subtracted in order to optimize the detection of lateral changes. If a particular PIR sensor is not sensitive enough to detect vertical position changes, a further detector may be added in order to optimize for vertical movement detection.

Subsequently, the differences between the various time points are calculated to derive a set of time durations T_(i,i+1)=t_(i+1)−t_i. These time duration parameters are for example sent to a server of the remote caregiver. The time duration parameters are tracked over time and are used to detect changes which may cause a notification for the remote caregiver or even trigger an alarm. The data may also be used to provide feedback to the elderly subject to give more insight into his/her behaviour.

PIR sensors are not the only possible sensors for detecting the presence and position of a subject. They provide a low cost solution but other arrangements are also possible.

One approach is to use vision based camera system with image processing used to identify and track the body position over time. The camera system may be triggered to operate when the subject is detected as being present, using a basic PIR detection sensor. Such image sensing may make use of markers placed on the subject, but as explained above it is preferred that no special measures need to be taken by the subject. The camera system may use depth based sensing to determine a depth from e.g. the ceiling, and thereby differentiate between sitting and standing.

Another approach is to detect presence within different horizontal sectors, which thereby indicate whether a standing or seating position is adopted. This approach has been proposed for detecting a fall of a subject in WO 2013/014578, for example in a bathroom. The sensors may again comprise PIR sensors, but other sensor types are possible such as ultrasound sensors, microwave sensors or millimetre wave sensors. By proving sensors at different heights, which detect movement within a horizontal band of space, posture information may be obtained.

As explained above, the sensor system is at least partially integrated into the structure of the toilet room. Some of the sensors may be worn by the subject. For example, by tracking the relative movement between different parts of the body, a sit or stand activity may be determined.

The invention may be implemented by any sensor arrangement or combination of sensors that provides sufficiently reliable discrimination between sitting and standing, so that timing measurements may be made.

The duration values may be used as a surrogate value for the real five times sit-to-stand test (SXSST). If a real SXSST measurement has been performed, the surrogate measure by the various timing duration values may then be calibrated using the real values.

The SXSST value may be achieved by extrapolating from individual toilet visits, for example by training the interpretation of the duration information using the real five times sit-to-stand test performance information obtained as a part of a test or calibration procedure. In this way, the monitoring system only needs to monitor individual sit and stand activities at each toilet visit, in order to extrapolate to a five times sit-to-stand test.

The monitoring system may also use the duration information collected from individual sit and stand activities in order to extrapolate to any sit-to-stand test involving one or more sit-to-stand and/or stand-to-sit activities, such as a two (2) times sit-to-stand test, three (3) times sit-to-stand test, four (4) times sit-to-stand test, ten (10) times sit-to-stand test, or as many times sit-to-stand test deemed appropriate.

However, the system may also allow the subject to perform a five times test not only as part of a system calibration but also periodically thereafter. The subject may either simply decide to perform a five times sit-to-stand activity every once in a while (e.g. weekly) whenever they remember. The system can then automatically detect this activity by detecting sequential sit and stand activities in a single toilet visit, and use the duration information as new calibration data. Alternatively, the system may have a mechanism for instructing the subject to perform a five times sit-to-stand activity, for example by communicating with a device worn by the subject, such as a smart phone or smart watch. Alternatively, a new calibration may be made during caregiver visits, so that the elderly subject does not need to remember to do so.

By using the same environment (i.e. the same toilet) for the calibration as for the activity monitoring, the results are more reliable.

FIG. 3 shows a method a method for monitoring the movement of a subject during toilet use, comprising in step 30 detecting the presence of the subject in a toilet room. In step 32 the timing t0-t5 of a set of events is recorded, based on the detection of the sitting or standing posture.

In step 34, the timing values are used to determine the duration of a stand-to-sit activity and the duration of a sit-to-stand activity.

In step 36, the duration information is stored for later output, or else output in real time, to an external device.

The system described above makes use of a controller 20 or processor for processing data.

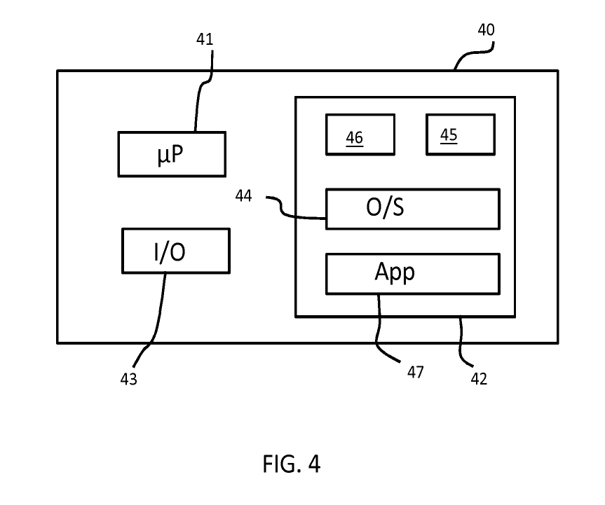

FIG. 4 illustrates an example of a computer 40 for implementing the controller or processor described above.

The computer 40 includes, but is not limited to, PCs, workstations, laptops, PDAs, palm devices, servers, storages, and the like. Generally, in terms of hardware architecture, the computer 40 may include one or more processors 41, memory 42, and one or more I/O devices 43 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 41 is a hardware device for executing software that can be stored in the memory 42. The processor 41 can be virtually any custom made or commercially available processor, a central processing unit (CPU), a digital signal processor (DSP), or an auxiliary processor among several processors associated with the computer 40, and the processor 41 may be a semiconductor based microprocessor (in the form of a microchip) or a microprocessor.

The memory 42 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 42 may incorporate electronic, magnetic, optical, and/or other types of storage media. Note that the memory 42 can have a distributed architecture, where various components are situated remote from one another, but can be accessed by the processor 41.

The software in the memory 42 may include one or more separate programs, each of which comprises an ordered listing of executable instructions for implementing logical functions. The software in the memory 42 includes a suitable operating system (O/S) 44, compiler 45, source code 46, and one or more applications 47 in accordance with exemplary embodiments.

The application 47 comprises numerous functional components such as computational units, logic, functional units, processes, operations, virtual entities, and/or modules.

The operating system 44 controls the execution of computer programs, and provides scheduling, input-output control, file and data management, memory management, and communication control and related services.

Application 47 may be a source program, executable program (object code), script, or any other entity comprising a set of instructions to be performed. When a source program, then the program is usually translated via a compiler (such as the compiler 45), assembler, interpreter, or the like, which may or may not be included within the memory 42, so as to operate properly in connection with the operating system 44. Furthermore, the application 47 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 43 may include input devices such as, for example but not limited to, a mouse, keyboard, scanner, microphone, camera, etc. Furthermore, the I/O devices 43 may also include output devices, for example but not limited to a printer, display, etc. Finally, the I/O devices 43 may further include devices that communicate both inputs and outputs, for instance but not limited to, a network interface controller (NIC) or modulator/demodulator (for accessing remote devices, other files, devices, systems, or a network), a radio frequency (RF) or other transceiver, a telephonic interface, a bridge, a router, etc. The I/O devices 43 also include components for communicating over various networks, such as the Internet or intranet.

When the computer 40 is in operation, the processor 41 is configured to execute software stored within the memory 42, to communicate data to and from the memory 42, and to generally control operations of the computer 40 pursuant to the software. The application 47 and the operating system 44 are read, in whole or in part, by the processor 41, perhaps buffered within the processor 41, and then executed.

When the application 47 is implemented in software it should be noted that the application 47 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The invention is of interest as part of a solution for elderly care services. The system may for example communicate with a wrist worn electronic patient file (storing an Electronic Health Record (EHR)) of the subject.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A monitoring system for monitoring the movement of a subject during toilet use, comprising:
a set of sensors for detecting the presence of the subject in a toilet room, and their sitting or standing posture;
a controller adapted to determine the duration of an individual stand-to-sit activity and the duration of an individual sit-to-stand activity, wherein the controller is further adapted to estimate a sit-to-stand test measurement for the subject based on the duration information, wherein the sit-to-stand test comprises a plurality of stand-to-sit activities and a plurality of sit-to-stand activities; and
a memory for storing the duration information or an output for outputting the duration information to an external device.

2. A system as claimed in claim 1, wherein the controller is adapted to estimate a five times sit-to-stand test measurement for the subject based on the duration information.

3. A system as claimed in claim 1, wherein the set of sensors comprises at least one PIR sensor.

4. A system as claimed in claim 3, wherein the at least one PIR sensor is for detecting presence in the toilet room and vertical movement.

5. A system as claimed in claim 1, wherein the set of sensors further comprises a microphone.

6. A system as claimed in claim 1, wherein the set of sensors further comprises a heart rate sensor and/or an accelerometer.

7. A system as claimed in claim 1, wherein the controller is further adapted to time the duration of a sitting activity and the duration of a standing activity before and after the sitting activity.

8. A monitoring installation, comprising a monitoring system as claimed in claim 1 installed in a toilet room.

9. A method for monitoring the movement of a subject during toilet use, comprising:
detecting the presence of the subject in a toilet room, and detecting their sitting or standing posture;
determining the duration of an individual stand-to-sit activity and the duration of an individual sit-to-stand activity;
storing the duration information or outputting the duration information to an external device; and
Estimating a sit-to-stand rest measurement for the subject based on the duration information, wherein the sit-to-stand test comprises a plurality of stand-to-sit activities and a plurality of sit-to-stand activities.

10. A method as claimed in claim 9, comprising detecting presence in the toilet room and detecting vertical movement.

11. A method as claimed in claim 9, comprising detecting sounds in the toilet room and/or detecting a heart rate of the subject and/or receiving movement information from an accelerometer device worn or carried by the subject.

12. A method as claimed in claim 9, comprising timing the duration of a sitting activity and the duration of a standing activity before and after the sitting activity.

13. A method as claimed in claim 9, comprising estimating a five times sit-to-stand test measurement for the subject based on the duration information.

14. A computer program comprising code means which is adapted, when said program is run on a computer, to implement the method of claim 9.

15. A computer program as claimed in claim 14 embodied on a computer readable medium.
